(19) United States
(12) Patent Application Publication
(10) Pub. No.: US 2023/0225432 A1
(43) Pub. Date: Jul. 20, 2023

(54) FACE MASK

(71) Applicant: Soliver AB, Helsingborg (SE)

(72) Inventor: Per KNUTSSON, Helsingborg (SE)

(73) Assignee: Soliver AB, Helsingborg (SE)

(21) Appl. No.: 18/246,954

(22) PCT Filed: Sep. 20, 2021
(86) PCT No.: PCT/SE2021/050900

§ 371 Date: Mar. 28, 2023

(30) Foreign Application Priority Data
Sep. 30, 2020 (SE) 2051136-6

(51) Int. Cl. A41D 13/11

(52) U.S. Cl. CPC A41D 13/1176; A41D 2300/328; A41D 2500/52

(57) ABSTRACT

This invention relates to a face mask (100, 200, 300) comprising a central area (40), comprising a fabric material (45), an elastic area (20), extending laterally at least partially from the central area (40) and comprising an elastic material (15), and being connected to the central area (40) at an interface section (30). Further, the elastic area (20) comprises an adhesive (11) provided on a bottom surface (12) of the elastic material (15) along at least a portion of a periphery of the face mask (100, 200, 300), forming an adhesive sealing profile (10). A space between the intersection section (30) and the sealing profile (10) comprising the elastic material (15) is an intermediate elastic area (25).

### TECHNICAL FIELD

The present invention relates to an elastic face mask.

### BACKGROUND

Face masks are used to protect the user from the environment, and face masks are commonly used for example by health care staff, in chemical and/or mining industries or is used to protect the user from air pollution and exhaust fumes.

The design of the face mask is adapted depending on what they aim to protect the user from. Face masks having variable complexity and degree of protection are available on the market. For instance, some face masks are strapless and may be attached directly onto the face of the user, while other comprise a valve for improved breathing ability.

Climate change caused by dangerously high emissions of fumes cause chronical lung diseases among the population in several countries globally, and the use of face masks in everyday life is common in many countries. Further, in health care, face masks are often required to prevent staff from being exposed to pathogens and to avoid the spread of body fluids from the mouth and nose of staff to patients.

In order for face masks to achieve and maintain proper function, the seal between the periphery of the mask and the face of the user is of importance. Leakage in the seal causes air containing pollutions, pathogens or other hazardous compositions to penetrates the barrier otherwise formed by the face mask, and its effect is diminished. Ruptures in the face mask seals are usually a result of movements in the face, e.g. caused when the user is talking.

WO2004/080219 discloses a face mask comprising a sealed face mask including a periphery with a seal surrounding the nostrils and the mouth, and which sticks to the skin. The adhesive of the seal may be a skin friendly adhesive or a skin unfriendly adhesive, and the seal includes a strip that may be elastomeric. With the skin unfriendly adhesive, the adhesive includes a strength sufficient to remove a first layer of skin from the face when the seal is pulled from the face. However, such removal is painful and harmful for the skin of the user and a skin friendly adhesive may result in a leakage caused by movements in the face.

EP1214896 relates to a face mask to control and improve the quality of inhaled air. The respiratory face mask comprises a flange and a filter layer, and the face masks are strapless and rely on a hydrogel adhesive for attachment and sealing or the face mask to the user. The adhesive may have elastic properties. However, none of the prior art documents present a well thought construction, which in turn has caused the face masks to lack a functionality which is industrially applicable and usable, and at the same time being cost effective.

Hence, there is a need for an improved elastic face mask solving the issue of seal leakages and mitigating the above mentioned problems within the art.

### SUMMARY OF THE INVENTION

Accordingly, the present invention preferably seeks to mitigate, alleviate or eliminate one or more of the above-identified deficiencies in the art and disadvantages singly or in any combination and solves at least the above mentioned problems by proposing a solution according to the appended independent claims. Advantageous embodiments are defined in the appended dependent claims.

In a first aspect, there is provided a face mask comprising a central area, comprising a fabric material, an elastic area, which extends laterally at least partially from the central area and comprises an elastic material and is connected to the central area at an interface section. The elastic area comprises an adhesive provided on a bottom surface of the elastic material along at least a portion of a periphery of the face mask, forming an adhesive sealing profile. A space between the intersection section and the sealing profile comprising the elastic material is an intermediate elastic area.

In one embodiment, the intermediate elastic area has an elasticity greater than that of the central area. Further, the intermediate elastic area may have an elasticity greater than that of the adhesive sealing profile.

Further advantages will be apparent from the detailed description as well as the appended dependent claims.

### BRIEF DESCRIPTION OF THE DRAWINGS

These and other aspects, features and advantages of which the invention is capable, will be apparent and elucidated from the following description of embodiments of the present invention, reference being made to the accompanying drawings, in which

FIG. 1A shows a user wearing a face mask according to the present disclosure;

FIG. 1B shows a longitudinal cross-section of the face mask worn by the user in

FIG. 1A;

FIG. 1C shows a cross-section of an elastic material comprised in the face mask according to the present disclosure;

FIG. 2A shows a second face mask according to the present disclosure;

FIG. 2B shows a user wearing the face mask shown in FIG. 2A; and

FIG. 3 shows yet another alternative example of a face mask according to the present disclosure.

### DETAILED DESCRIPTION OF THE EMBODIMENTS

Embodiments of the present invention will be described in more detail below with reference to the accompanying drawings in order for those skilled in the art to be able to carry out the invention. The invention may, however, be embodied in many different forms and should not be construed as limited to the embodiments set forth herein. Rather, these embodiments are provided so that this disclosure will be thorough and complete, and will fully convey the scope of the invention to those skilled in the art. The embodiments do not limit the invention, but the invention is only limited by the appended patent claims. Furthermore, the terminology used in the detailed description of the particular embodiments illustrated in the accompanying drawings is not intended to be limiting of the invention.

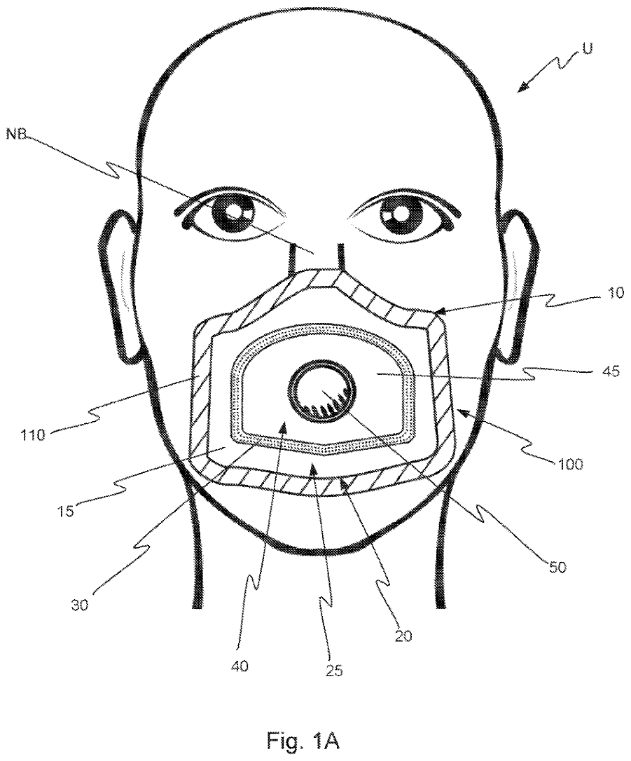

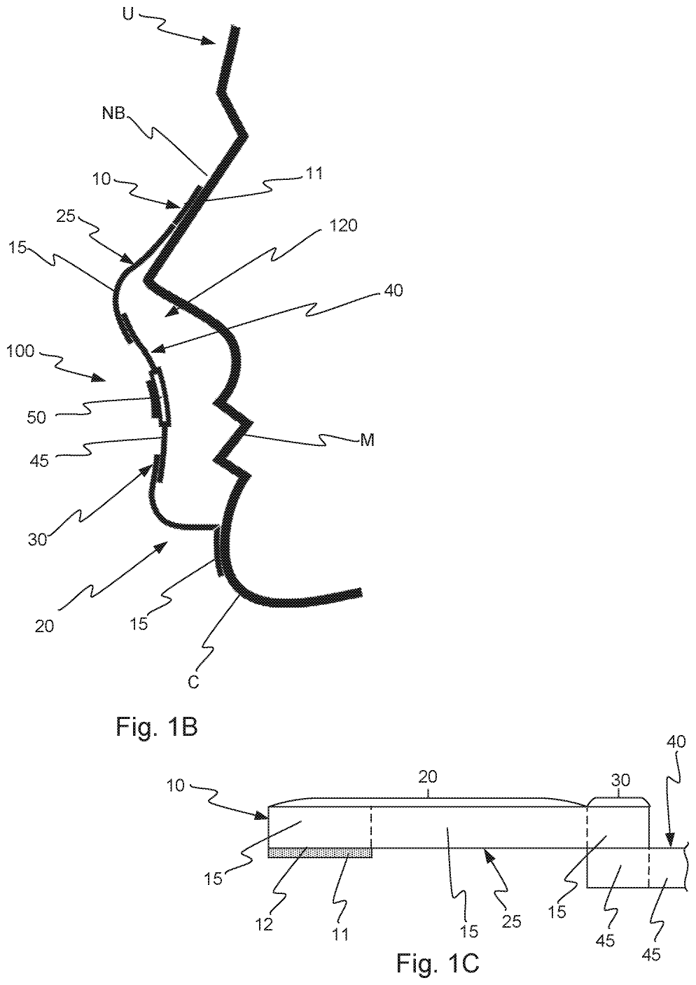

With reference to the FIGS. 1A-3, three embodiments of face masks 100, 200, 300 are shown. The three face masks 100, 200, 300 all comprise four main areas 10, 20, 30, 40, arranged as follows:
a central area 40, comprising a fabric material 45;
an elastic area 20, arranged and extending laterally from the central area 40 and formed from an elastic material 15. The elastic material 15 is connected to the central area 40 at an interface section 30; and
the elastic area 20 comprises an adhesive matrix 11 (also referred to as an adhesive 11) provided on a bottom surface 12 of the elastic material 15 along at least a portion of a periphery 110, 210, 310 of the face mask 100, 200, 300 thus forming an elastic adhesive sealing profile 10 and an intermediate area 25 located between said sealing profile 10 and the interface.

Hence, an elastic intermediate area 25 is formed between the interface section 30 and the elastic adhesive sealing profile 10, which is also referred to as the sealing profile 10 herein.

FIGS. 1A and 1B show a user U wearing the face mask 100, covering the mouth M (shown in FIG. 1B) and nose area of the user U. The face mask 100 is a strapless face mask 100, which is attached directly onto the skin of the user U by means of the adhesive matrix 11 of the elastic material 15 in the sealing profile 10. In the face mask 100, the elastic material 15 and the sealing profile 10 extend around the periphery 110 of the face mask 100.

The mouth M and a portion of the nose, up to a certain height of the nasal bone NB, represent a critical area which is of importance to cover by a face mask 100. To cover the mouth M and nose prevents pollutions in the air from the environment, such as pathogens or dust, to be inhaled by the user U, and prevents body fluids to spread from the user U to the surroundings. As shown in FIGS. 1A and 1B, the face mask 100 covers such critical area with sufficient margin.

The face mask 100 comprises the four main areas 10, 20, 30, 40 and a breathing valve 50. The elastic area 20 comprises the elastic material 15, which is divided into the elastic intermediate area 25 and the sealing profile 10. The outer periphery 110 of the elastic material 15 (and the face mask 100) comprises the elastic sealing profile 10, in which the elastic material 15 is provided with the adhesive matrix 11 (also referred to herein as an adhesive 11) on the bottom surface 12 thereof. The adhesive 11 attaches to the skin of the user U such that the face mask 100 does not need any fastening straps.

Centrally disposed, next to the elastic sealing profile 10, the face mask 10 comprises the intermediate area 25 formed from the same elastic material 15 as used for the sealing profile 10. However, the elastic material 15 of the intermediate area 25 is not provided with an adhesive on its bottom surface facing the user U. Hence, the dashed area of the sealing profile 10 in FIG. 1A indicates the area of the elastic material 15 which is provided with the adhesive matrix 11 on the bottom surface 12 thereof (as shown in FIG. 1C). As shown in FIG. 1B, the intermediate elastic area 25 is separated from and not in contact with the face of the user U. Hence, there is a free passage between the face of the user U and the intermediate elastic area 25 due to the spacing distance there between.

FIG. 1C shows a cross-section of mainly the elastic area 20 comprising the elastic material 15 forming the intermediate area 25 and the sealing profile 10. An artificial boundary between the sealing profile 10 and the intermediate area 25 is indicated by the dashed vertical line in FIG. 1C. As shown in FIG. 1C, the elastic adhesive sealing profile 10 comprises the adhesive 11 on the bottom surface 12. The boundary between the elastic area 20 and the interface section 30 is also indicated by a dashed line in FIG. 1C. The elastic material 15 is arranged on top of the fabric material 45 in the interface section 30. Further, a portion of the central area 40 comprising the fabric material 45 is shown in FIG. 1C. The boundary between the interface section 30 and the central area 40 is also indicated by a dashed line. Observe that FIG. 1C is not in scale.

The adhesive matrix 11 adheres to the skin of the user U such that the face mask 100 does not require any attachment straps and also helps forming barrier between the face mask 100 and the user's skin.

Optionally, a second elastic material may be used to form the intermediate area 25. Such material should exhibit similar elastic properties, preferably having equally elastic properties, to that of the elastic material 15 used to form the sealing profile 10. In the case of the use of a second elastic material, the sealing profile 10 and the intermediate area 25 should be bonded in a way such that no rigid or stiff interface is formed. Such interface would decrease the possibility if the sealing profile 10 to compensate for facial movements. Experiments have shown that stiff joints between the sealing profile 10 and the intermediate area 25 formed from two different elastic materials results in leakage in the critical area of the nasal region. The interaction between the sealing profile 10 and the intermediate area 25 will be explained more in the following.

Further with reference to FIGS. 1A and 1B, the intermediate area 25 (and thus the elastic material 15) is joined with the central area 40 in the interface area 30, either by sewing, by an adhesive (e.g. a glue), by a combination of sewing and an adhesive, or any other suitable attachment technique known in the art. Preferably, the intermediate area 25 is attached to the central area 40 by sewing, as indicated by the dotted lines in the interface area 30 in FIG. 1A. The interface area 30 is also referred to as the interface section 30 herein.

In the interface section 30, the elastic material 15 forming the intermediate area 25 overlap with the fabric material 45 constituting the central area 40. The fabric material 45 is preferably a fibre cloth 45 (such as ventilated cotton cloth), which exhibits little or no elasticity. Since the fibre cloth 45 has little or no elasticity, the interface area 30 also exhibits little or no elasticity.

In FIG. 1A and 1B, the face mask 100 is provided with the valve 50 arranged in the central area 40 in the fibre cloth 45. The valve 50 may be a plastic filter valve which is fitted in the face mask 100 using a snip-fit mechanism, which requires a relatively non-elastomeric material to ensure a secure attachment. If the central material 40 is made of e.g. the elastic material 15, the elastic material 15 will stretch during an attempt to fasten the valve 50. However, the central area 40 made of a fibre cloth 45 is less elastic and enables a secure and durable attachment of the valve 50 therein, and which can withstand the force of attaching a valve 50 with a snap-fit mechanism. If the central area 40 was made of a material with elastic properties, the valve 50 may come off when the material is compressed in the direction of the thickness of the material 45 and it is difficult to ensure a secure attachment.

FIG. 1B shows a longitudinal cross-section of the face mask 100 attached to the user U. The bottom surface 12 (shown in FIG. 1C) of the sealing profile 10 is provided with an adhesive 11 forming a tight seal between the face mask 100 and the face of the user U, FIG. 1B showing the attachment of the face mask 100 to the user's nasal bone NM and chin C.

The intermediate area 25 adjacent to the sealing profile 10 is arranged centrally of the periphery 110 and is connected to the central area 40 in the interface section 30. In FIG. 1B, the fabric material 45 is disposed closer to the face of the user U than the elastic material 15 in the interface section 30. However, the central area 40 and the intermediate area 25 may be arranged oppositely, the elastic material 15 being arranged closer to the user's face. The valve 50 is connected to the fabric material 45 of the central area 40.

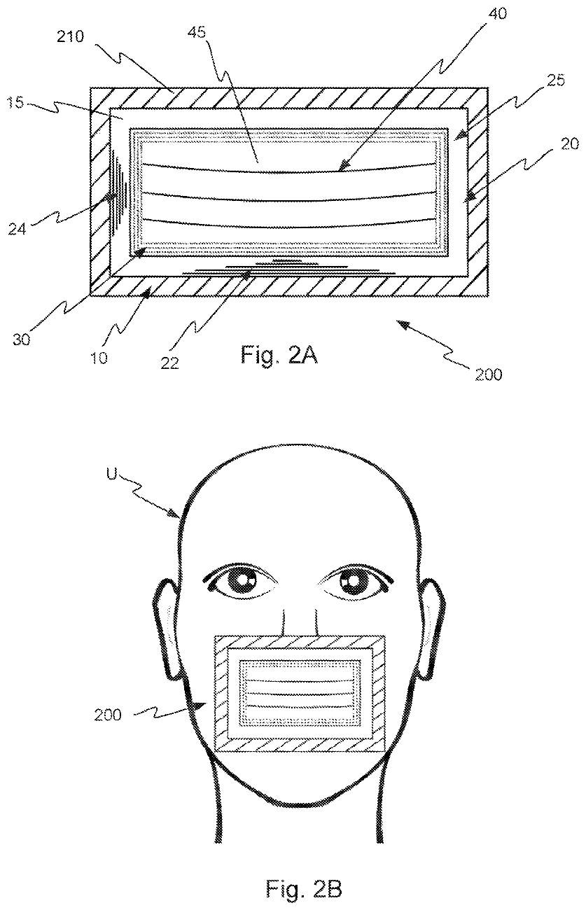

FIG. 2A shows a rectangular shaped face mask 200 comprising the same four regions as the face mask 100 shown in FIGS. 1A and 1B, namely the elastic area 20 comprising the sealing profile 10 and the intermediate area 25, the interface section 30 and the central area 40. The sealing profile 10 and the intermediate area 25 are formed from the elastic material 15 and the elastic area 20 exhibits the same cross-section as shown in FIG. 1C (i.e. the sealing profile 10 is provided with an adhesive matrix 11 on the bottom surface 12 facing the user U when attached to the face, while the intermediate area 25 does not comprise such adhesive 11). The elastic material 15 of the intermediate area 25 is stitched to the central area 40 in the interface section 30, as indicated by the dashes in the interface area 30. The central area 40 is formed from a fabric material 45, which has been folded into a plurality of creases, as shown by the vertical lines in the central area 40 in FIGS. 2A and 2B. Preferably, in this embodiment the fabric material 45 is a cotton material.

The rectangular shape of the face mask 200 facilitates the production and is cheap to produce. Also in the face mask 200, a thickness of the elastic material 15 is in the range of 0.1 to 1.5 mm, preferably between 0.1 and 0.4 mm. A thinner elastic material 15 also decreases manufacturing costs. Even though the design of the face mask 200 is simple and easy to manufacture, the sealing profile 10 and the intermediate area 25 comprising the same elastic material 15 result in a tight seal also in the rectangular design shown in FIG. 2A.

Further, two stretch areas 22 and 24 in the intermediate area 25 are shown in FIG. 2A. The lines in the stretch areas 22, 24 indicate a non-linear stretching of the elastic material 15. Closer to the interface section 30, the elastic material 30 is less stretched than adjacent to the sealing profile 10, since the interface area 30 is stiff, while the sealing profile 10 is elastic. The degree of stretching of the elastic material 15 is indicated by the lines in the stretch areas 22, longer lines indicating more stretching.

An exemplary total width of the elastic material 15 in the face mask 200 is 25 mm (along all four sides). In such case, the elastic material 15 is disposed as 10 mm as the sealing profile 10, 10 mm as the intermediate area 25 and 5 mm in the interface section 30.

With reference to FIG. 2B, the face mask 200 is worn by the user U.

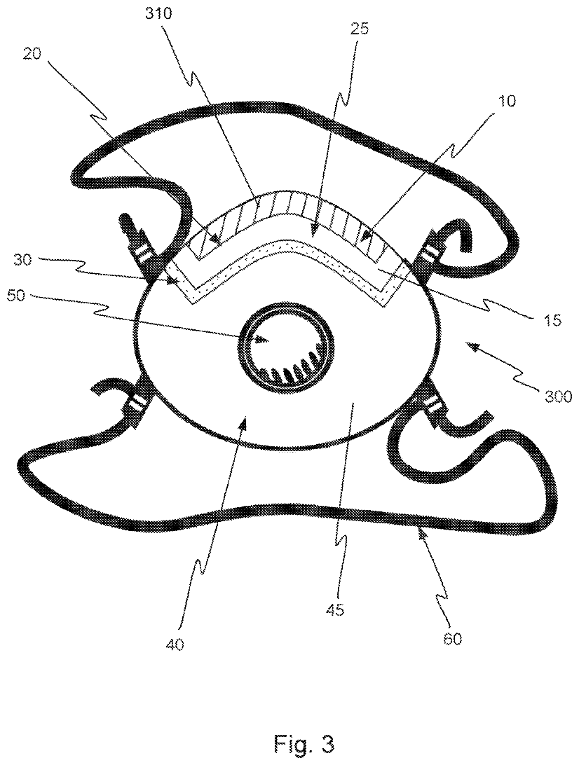

FIG. 3 discloses a face mask 300 according to another embodiment disclosed herein, in which the sealing profile 10 is arranged partially along the circumference of the face mask 300. The sealing profile 10 extends along an outer edge of the face mask 300 in the critical nasal bone area NB, which usually is the most complicated area in the face of a user U (shown in FIGS. 1A, 1B and 2B) to achieve a tight seal.

The sealing profile 10 comprises an elastic material 15 and an adhesive matrix 11 arranged on a bottom surface 12 of the profile 10, as shown with reference to FIG. 1C. The elastic material 15 extends from the sealing profile 10 to form an intermediate area 25, which do not comprise the adhesive 11. A central area 40 comprises a fabric material 45, preferably a fibre cloth 45 and a valve 50. The fibre cloth 45 is connected to the elastic material 15 of the intermediate area 25 in an interface section 30. Further, the face mask 300 comprises adjustable straps 60, which helps to hold the face mask 300 in place during use.

Other face masks in the art provides solutions where a nasal clip is arranged in an attempt to achieve a seal between face mask and face of the user. However, such attempt is non-reliable and does not provide a tight seal, and is further easily affected negatively due to nasal movements.

During use, the outer peripheral sealing profile 10 is in direct contact with the face of the user U through the adhesive 11. Hence, all facial movements caused by the user U place high demands on the sealing profile 10 to prevent leakage. Face masks known in the art struggle to keep the barrier between face mask and skin intact.

However, the face mask 100 of the present disclosure include the intermediate area 25 formed from the elastic material 15. Since the material 15 of intermediate area 25 will stretch upon stress and strain, an effect of the use of the elastic material 15 for the intermediate area 25 is that the adhesive 11 of the sealing profile 10 will not detach from the skin of the user U even upon vigorous facial movements. In addition, since the adhesive 11 in the sealing profile 10 in combination with the elastic material 15 in the intermediate elastic area 25 is equally effective over the entire facial contour, hot air is prevented from passing between the nose and the face mask 100, 200, 300, which in turn may cause condensation on glasses or protective glasses used by the user U. This issue is a major disadvantage with face masks known in the art, which fail to prevent the formation of such condensation, due to non tight seals between the nose and the face of the user. This will be explained more in the following.

Since the fibre cloth 45 forming the central area 40 has limited stretchability or is non-stretchable, the elastic material 15 has decreased elasticity in the interface section 30 where it is overlapping with the central area material 45 (shown in FIG. 1B). However, the intermediate area 25 between the interface section 30 and the sealing profile 10 is stretchable, which facilitates the sealing profile 10 to exhibit its full elastic behaviour without the risk of detaching from the face of the user U. Since the sealing profile 10 and the intermediate area 25 are formed from the same material 15, there is no stiff interface between them. Therefore, the intermediate area 25 will compensate for facial movements and thereby decrease the transfer of drag and tensions to the sealing profile 10. This means that even in the case of substantial facial movements, such as jaw movements, will not cause the adhesive matrix 11 on the bottom surface 12 of the elastic sealing profile 10 to come off the user's face. Hence, a tight barrier can be maintained and the risk of leakage of ambient air into the face mask 100 is decreased.

Width of the sealing profile 10 and the intermediate area 25 is also important for the interaction there between. Another effect of using the elastic material 15 also in the intermediate area 25 is that a smaller contact area between the skin of the user U and the sealing profile 10 is required to maintain a fluid tight seal there between. To allow an optimised elasticity interaction, the width of the sealing profile 10 and the intermediate area 25 is each preferably 2 to 25 mm. More preferably, the intermediate area 25 has at least the same width as the sealing profile 10. This width range allows for enough wiggle room such that the sealing profile 10 is not pulled to such extent that it detaches from the face of the user U upon facial movements. Experiments performed on hydrocolloid adhesive dressings have shown this to be true. In addition, the adhesiveness of the adhesive 11 can be decreased, and still maintain the sealed barrier.

Preferably, a width of the interface section 30 is 25-35% of a width of the intermediate area 25. Hence, 65-75% of the width of the elastic material 15 in the intermediate area 25 will be able to provide elastic movements.

If the sealing profile 10 were to be attached directly to a less elastic or non-elastic area, such as the central area 40, and the intermediate area 25 was excluded, facial movements would cause the stiffer material 45 to pull the sealing profile 10. Hence, tensions in the sealing profile 10 would result in a leakage between the sealing profile 10 and the user's face.

Instead, in the face mask 100, the intermediate area 25 provides even more elasticity than the sealing profile 10 even when formed from the same elastic material 15, since the intermediate area 25 is not provided with an adhesive 11 and since there is a clearance 120 between the face of the user U and the second elastic material 25 (shown in FIG. 1B). Thus, the elastic material 15 in the intermediate area 25 will relieve the sealing profile 10 from tensions, drag and movements in the face mask 100. The clearance 120 is of particular importance during a substantial opening of the mouth M.

In face masks comprising a sealing profile with an adhesive attaching said mask to the skin of the user, there is an interaction between the adhesiveness of the adhesive matrix and the experienced feeling upon removal of the face mask. Higher adhesiveness results in higher pain upon removal. The width of the sealing profile also affects the level of experienced pain during detachment. Hence, the face mask 100 disclosed herein is designed such that the user U does not experience such pain. By extending the elastic material 15 beyond the sealing profile 10 to form the intermediate area 25, the adhesive matrix 11 can have an adhesiveness sufficiently high to adhere to the skin of the user U, but sufficiently low to avoid painful removal of the face mask 100.

The sealing profile 10 and the intermediate area 25 preferably have a thickness in the range of 0.1-1.5 mm, more preferably between 0.1-0.4 mm. The adhesiveness of the sealing profile 10 and the interface region 30 allows for a thin thickness of the elastic material 15, improving the elastic characteristics, while simultaneously using the more course central area 40 to bear the valve 50 without any issues. The thickness of the elastic material 15 also affects its elasticity. A thinner elastic material 15 is more formable with respect to irregularities of the facial contour and during movements, since it is more stretchable. A thickness of the sealing profile 10 is preferably in the range of 0.1-1.5 mm, more preferred within 0.1-0.4 mm. Further, a thin thickness is advantageous in that it provides for a cost effective production.

In hospital environments, specifically in for instance intensive care units, the user U will wear the face mask 100 during long periods at the time. The face mask 100 must therefore provide a sustainable barrier to the ambient air and surroundings to prevent leakage between the face mask 100 and the user's skin.

Preferably, the elastic material 15 is an elastomer which may be stretched to at least its double length, and then return to its resting state without suffering from stress relaxation. Hence, the elastic material 15 is formable. The elastic material 15 may be a thermoplastic elastomer (such as a thermoplastic olefin (TPO) based material, a polyurethane (TPU) based material or a hydrogel), a synthetic rubber sheet, or a silicone sheet. Preferably, the elastomer is a biodegradable elastomer. Preferably, the elastic material 15 and the adhesive 11 used on the bottom surface 12 thereof are medical grade materials. This reduces the risk of side effects for the user U, such as rashes, wounds or skin irritation.

The elastic material 15 and the adhesive matrix 11 on the bottom surface 12 thereof may also provide an anti-septic barrier. In such case, an anti-septic is added to the adhesive matrix 11 to prevent pathogens from penetrating the barrier formed between the user U and the attached face mask 100.

Further, the invention has mainly been described with reference to a few embodiments. However, as is readily understood by a person skilled in the art, other embodiments than the ones disclosed above are equally possible within the scope of the invention, as defined by the appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1.-17. (canceled)

18. A face mask comprising:
a central area including a fabric material;
an elastic area extending laterally at least partially from the central area, the elastic area including an elastic material connected to the central area at an interface section; and
wherein the elastic area includes an adhesive provided on a bottom surface of the elastic material along at least a portion of a periphery of the face mask to form an adhesive sealing profile; and
wherein a space between the interface section and the adhesive sealing profile including the elastic material is an intermediate elastic area.

19. The face mask according to claim 18, wherein the intermediate elastic area has an elasticity greater than that of the central area.

20. The face mask according to claim 18, wherein the intermediate elastic area has an elasticity greater than that of the adhesive sealing profile.

21. The face mask according to claim 18, wherein the elastic material is selected from the group consisting of a thermoplastic elastomer, a hydrocolloid, and a medical grade synthetic rubber sheet or silicone sheet.

22. The face mask according to claim 18, wherein the adhesive is made of a hydrogel, or a plaster adhesive matrix.

23. The face mask according to claim 18, wherein the elastic material is stretchable to a length being at least twice its extension in its resting state in all three dimensions, and return to its resting state upon relaxation.

24. The face mask according to claim 18, wherein a width of the elastic area is in a range of 10 to 50 mm.

25. The face mask according to claim 18, wherein a width of the adhesive sealing profile is in a range of 2 to 25 mm.

26. The face mask according to claim 18, wherein a width of the intermediate elastic area is in a range of 2 to 25 mm.

27. The face mask according to claim 18, wherein a width of the interface section is 25-35% of a width of the intermediate elastic area.

28. The face mask according to claim 18, wherein a thickness of the elastic material is in a range of 0.1 to 1.5 mm.

29. The face mask according to claim 18, wherein the elastic area extends around the entire central area of the face mask.

30. The face mask according to claim 29, wherein the adhesive provided on the bottom surface of the elastic material extends around the periphery of the face mask, such that the adhesive sealing profile circumvents the face mask.

31. The face mask according to claim 18, further comprising a valve arranged in the central area.

32. The face mask according to claim 31, wherein the valve is attached to the central area by a snap-fit mechanism.

33. The face mask according to claim 18, wherein the adhesive and/or the elastic material further comprises an anti-septic agent.

34. The face mask according to claim 18, wherein the elastic material is connected to the fabric material of the central area in the interface section by sewing, through an adhesive, or a combination thereof.

35. The face mask according to claim 18, wherein the intermediate elastic area is separated from and not in contact with the face of a user.
